(19) United States
(12) Patent Application Publication
(10) Pub. No.: US 2009/0270971 A1
(43) Pub. Date: Oct. 29, 2009

(54) Prosthesis Fixation Apparatus and Methods

(75) Inventors: Jia Hua Xiao, Santa Rosa, CA (US); Trevor Greenan, Santa Rosa, CA (US); Walter Bruszewski, Guerneville, CA (US); David Gray, Windsor, CA (US); Mark L. Stiger, Windsor, CA (US); Morgan House, Newfields, NH (US); Matthew Rust, North Vancouver (CA)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/109,076

(22) Filed: Apr. 24, 2008

(51) Int. Cl. A61F 2/06

(52) U.S. Cl. 623/1.14; 623/1.13

(57) ABSTRACT

A tubular prosthesis comprises a tubular graft; and an undulating stent having a plurality of apexes, a first end defined at least in part by a first group of the apexes, and a second end defined at least in part by a second group of the apexes, the first group of apexes being pivotally attached to the tubular graft so as to form a plurality of circumferentially arranged hinges about which the stent can pivot so that the second group of apexes can move between a position where they are inside the tubular graft and a position where they are outside the tubular graft. In one embodiment a tubular prosthesis comprises a tubular graft having a first end margin, a second end margin and a central portion therebetween; and an undulating stent having a plurality of apexes, a first end defined at least in part by a first group of the apexes, and a second end defined at least in part by a second group of the apexes, the undulating stent being secured to the tubular graft in a manner such that it can be inverted to extend generally in the same direction as the tubular graft with one end thereof forming an end of said tubular prosthesis and pointing away from the central portion of the tubular graft.

### FIELD OF THE INVENTION

The invention relates to prosthesis fixation and/or sealing in a passageway in a human body such as an artery.

### BACKGROUND OF THE INVENTION

Tubular prostheses such as stents, grafts, and stent-grafts (e.g., stents having an inner and/or outer covering comprising graft material and which may be referred to as covered stents) have been used to treat abnormalities in passageways in the human body. In vascular applications, these devices often are used to replace or bypass occluded, diseased or damaged blood vessels such as stenotic or aneurysmal vessels. For example, it is well known to use stent-grafts, which comprise biocompatible graft material (e.g., Dacron® or expanded polytetrafluoroethylene (ePTFE)) supported by a framework (e.g., one or more stent or stent-like structures), to treat or isolate aneurysms. The framework provides mechanical support and the graft material or liner provides a blood barrier.

Aneurysms generally involve abnormal widening of a duct or canal such as a blood vessel and generally appear in the form of a sac formed by the abnormal dilation of the duct or vessel wall. The abnormally dilated wall typically is weakened and susceptible to rupture. Aneurysms can occur in blood vessels such as in the abdominal aorta where the aneurysm generally extends below the renal arteries distally to or toward the iliac arteries.

In treating an aneurysm with a stent-graft, the stent-graft typically is placed so that one end of the stent-graft is situated proximally or upstream of the diseased portion of the vessel and the other end of the stent-graft is situated distally or downstream of the diseased portion of the vessel. In this manner, the stent-graft extends through the aneurysmal sac and beyond the proximal and distal ends thereof to replace or bypass the weakened portion. The graft material typically forms a blood impervious lumen to facilitate endovascular exclusion of the aneurysm.

Such prostheses can be implanted in an open surgical procedure or with a minimally invasive endovascular approach. Minimally invasive endovascular stent-graft use is preferred by many physicians over traditional open surgery techniques where the diseased vessel is surgically opened and a graft is sutured into position such that it bypasses the aneurysm. The endovascular approach, which has been used to deliver stents, grafts, and stent grafts, generally involves cutting through the skin to access a lumen of the vasculature. Alternatively, luminal or vascular access may be achieved percutaneously via successive dilation at a less traumatic entry point. Once access is achieved, the stent-graft can be routed through the vasculature to the target site. For example, a stent-graft delivery catheter loaded with a stent-graft can be percutaneously introduced into the vasculature (e.g., into a femoral artery) and the stent-graft delivered endovascularly across the aneurysm where it is deployed.

When using a balloon expandable stent-graft, balloon catheters generally are used to expand the stent-graft after it is positioned at the target site. When, however, a self-expanding stent-graft is used, the stent-graft generally is radially compressed or folded and placed at the distal end of a sheath or delivery catheter. Upon retraction or removal of the sheath or catheter at the target site, the stent-graft self-expands.

More specifically, a delivery catheter having coaxial inner and outer tubes arranged for relative axial movement therebetween can be used and loaded with a compressed self-expanding stent-graft. The stent-graft is positioned within the distal end of the outer tube (sheath) and in front of a stop fixed to the inner tube. Once the catheter is positioned for deployment of the stent-graft at the target site, the inner tube is held stationary and the outer tube (sheath) withdrawn so that the stent-graft is gradually exposed and expands. The inner tube or plunger prevents the stent-graft from moving back as the outer tube or sheath is withdrawn. An exemplary stent-graft delivery system is described in U.S. Pat. No. 7,264,632 to Wright et al. and is entitled Controlled Deployment Delivery System, the disclosure of which is hereby incorporated herein in its entirety by reference.

Regarding proximal and distal positions referenced herein, the proximal end of a prosthesis (e.g., stent-graft) is the end closer to the heart (by way of blood flow) whereas the distal end is the end farther away from the heart during deployment. In contrast, the distal end of a catheter is usually identified as the end that is farthest from the operator, while the proximal end of the catheter is the end nearest the operator.

Although the endolumenal approach is much less invasive, and usually requires less recovery time and involves less risk of complication as compared to open surgery, among the challenges with this approach are fixation, migration, and sealing of the prosthesis. For example, the outward spring force of a self-expanding stent-graft may not be sufficient to prevent migration. This problem can be exacerbated when the vessel's fixation zone significantly deviates from being circular. And when there is a short landing zone, for example, between an aortic aneurysm and a proximal branching artery (e.g., one of the renal arteries, or the carotid or brachiocephalic artery), small deviations in sizing or placement may result in migration and or leakage.

Current endovascular devices incorporate stent-graft over-sizing to generate radial force for fixation and/or sealing and some have included fixation mechanisms comprising radially extending members such as tines, barbs, hooks and the like that engage the vessel wall to reduce the chance of migration. In some abdominal aortic aneurysm applications, a suprarenal stent and hooks are used to anchor the stent-grafts to the aorta. However, abdominal aortic aneurysm stent-grafts typically require an anchor or landing zone of about 10-15mm to achieve the desired fixation and seal efficacy. In some cases, such an anchoring or landing zone does not exist due to diseased vasculature or challenging anatomy. In these cases, an endolumenal device (e.g., a graft or stent-graft) is placed in the vessel such that it extends beyond the landing zone and the adjacent branch or branch vessels and a secondary device (e.g., a branch graft or branch stent-graft) placed through a fenestration or side opening in the main device and into a branch vessel. One example is when an aortic abdominal aneurysm is to be treated and its proximal neck is diseased or damaged to the extent that it cannot support a connection and/or seal with a prosthesis. In this case, grafts or stent-grafts have been provided with fenestrations or openings formed in their side wall below a proximal portion thereof to perfuse the branch vessels and a branch graft or stent-graft delivered through the fenestration and coupled to the main graft or stent-graft.

One staple approach to improve fixation is described in copending, co-owned U.S. Patent Application Publication 2007/0219627 by Jack Chu et al, which was filed on Mar. 17, 2006 and is entitled Prosthesis Fixation Apparatus and Methods, involves delivering a fastener having a proximal piercing end portion and a distal piercing end portion to a site where a prosthesis having a tubular wall has been placed in the passageway, which has a wall, advancing the proximal piercing end portion beyond the prosthesis, penetrating the proximal piercing end portion into the wall of the passageway without passing the proximal piercing end portion through the tubular wall of the prosthesis, and passing the distal piercing end portion through the tubular wall of the prosthesis and into the wall of the passageway. Other approaches to improve fixation and/or sealing between the prosthesis and an endolumenal wall have included using adhesives and growth factor (see e.g., copending, co-owned U.S. Patent Application Publication 2007/0233227 by Trevor Greenan, which was filed on Mar. 30, 2006 and is entitled Prosthesis with Coupling Zone and Methods. Another fixation approach described in copending, co-owned U.S. patent application Ser. No. 11/736,453 by Jia Hua Xaio et al, filed Apr. 17, 2007 and entitled Prosthesis Fixation Apparatus and Methods, involves endolumenally advancing fasteners to a plurality of sites within a prosthesis such as a stent-graft and passing the fasteners from an inner surface of the prosthesis through the prosthesis and a wall of the passageway to which the prosthesis is to be secured. In one embodiment, the fasteners are deployed simultaneously and in another embodiment they are deployed serially. Further prosthesis fixation apparatus is described in copending, co-owned U.S. patent application Ser. No. 11/928,379 by Jia Hua Xaio, filed Oct. 30, 2007 and entitled Prosthesis Fixation Apparatus and Methods.

There remains a need to develop and/or improve seal fixation and/or sealing approaches for endolumenal or endovascular prosthesis placement.

### SUMMARY OF THE INVENTION

The present invention involves improvements in prosthesis fixation. In one embodiment according to the invention, a tubular prosthesis comprises a tubular graft having a first end margin, a second end margin and a central portion therebetween; and an undulating stent having a plurality of apexes, a first end defined at least in part by a first group of the apexes, and a second end defined at least in part by a second group of the apexes, the undulating stent being secured to the tubular graft in a manner such that it can be inverted to extend generally in the same direction as the tubular graft with one end thereof forming an end of said tubular prosthesis and pointing away from the central portion of the tubular graft.

In another embodiment according to the invention, a tubular prosthesis delivery system comprises a sheath having a distal deployment end and a proximal end; a radially compressed stent-graft, which has a first end and a second end and is slidably disposed in the sheath and further includes an undulating stent having a plurality of apexes, a first end of the stent being defined at least in part by a first group of the apexes, and a second end of the stent being defined by at least in part by a second group of the apexes, the undulating stent being inverted with the second group of apexes directed toward the distal deployment end of the sheath.

In another embodiment according to the invention, a method of delivering a tubular prosthesis in a vessel in a human patient comprises delivering a tubular prosthesis having an inner surface, an outer surface, and an inverted stent forming the leading end of the prosthesis as it is delivered to a target site in a human vessel and deploying the prosthesis such that the inverted stent folds back over one of the inner and outer surfaces of the tubular prosthesis.

In another embodiment according to the invention, a method of coupling a first tubular prosthesis in a branch vessel to a second tubular prosthesis in a vessel from the branch vessel branches comprises delivering a first tubular prosthesis, which is restrained in a sheath and has a leading end and a trailing end, which includes an inverted stent, through a fenestration in a second tubular prosthesis, which is positioned in a first vessel, and into a second vessel that branches from the first vessel; positioning the inverted stent inside the first tubular prosthesis; and retracting the sheath to release the first tubular prosthesis and allow the trailing end to move radially outward against an inner surface of the second prosthesis adjacent the branch vessel to form a seal between the first and second prostheses.

In another embodiment according to the invention, a tubular prosthesis comprises a tubular graft; and an undulating stent having a plurality of apexes, a first end defined at least in part by a first group of the apexes, and a second end defined at least in part by a second group of the apexes, the first group of apexes being pivotally attached to the tubular graft so as to form a plurality of circumferentially arranged hinges about which the stent can pivot so that the second group of apexes can move between a position where they are inside the tubular graft and a position where they are outside the tubular graft.

The above is a brief description of some deficiencies in the prior art and advantages of embodiments according to the present invention. Other features, advantages, and embodiments according to the present invention will be apparent to those skilled in the art from the following description and accompanying drawings, wherein, for purposes of illustration only, specific embodiments are set forth in detail.

### BRIEF DESCRIPTION OF THE DRAWINGS

FIG. 1A illustrates one embodiment of a stent-graft with an inverting or invertible stent according to the invention.

FIG. 1B diagrammatically illustrates the stent-graft of FIG. 1A with the inverting or invertible stent inverted and radially compressed for loading into a stent-graft delivery sheath.

FIG. 1C diagrammatically illustrates the stent-graft of FIG. 1A radially compressed and loaded in a stent-graft delivery sheath and the inverting or invertible stent inverted and restrained in a tube.

FIG. 1D diagrammatically illustrates a delivery or release approach for the stent-graft of FIG. 1A where the leading end of the stent is restrained in an inverted configuration while the remainder of the stent-graft is radially expanded so that when the restraint is removed, the inverted stent returns or springs back to the position along the inner surface of the stent-graft as shown in FIG. 1A.

FIG. 1E is an end view of the stent-graft of FIG. 1D.

FIG. 1F illustrates another configuration of the stent of FIG. 1A after a delivery approach where the inverted stent was unrestrained as it was deployed from the stent-graft delivery sheath in a manner such that it returns to a position along the outer surface of the stent graft.

FIGS. 2A-D illustrate one embodiment for delivering the stent-graft of FIG. 1A, where FIG. 2A shows the stent-graft radially compressed in a sheath as it is delivered to a desired site, FIG. 2B shows partial deployment of the stent-graft while maintaining the distal end of the inverting or invertible stent restrained in an inverted state, FIG. 2C shows partial release of the inverted stent, and FIG. 2D shows full release of the leading end of the inverted stent and the inverting stent after it has returned to a position as shown in FIG. 1A.

FIGS. 2E-G diagrammatically illustrate deployment of the inverted stent of FIG. 1A with optional hooks, where FIG. 2E shows the inverted stent released, FIG. 2F shows the stent inside the stent-graft and the hooks penetrating the stent-graft, and FIG. 2G shows the stent along the inner surface of the stent-graft and the hooks fully engaged through the vessel wall where the stent-graft was deployed.

FIGS. 2H-J diagrammatically illustrate deployment of another stent-graft embodiment according to the invention, which also includes an invertible stent hingedly coupled to a tubular graft where FIG. 2H shows the stent restraint of FIGS. 2E-G being retracted after partial release of the invertible stent to assist in pivoting the stent about its circumferentially oriented hinge points to an inverted position within the tubular graft of the stent-graft, FIG. 2I shows further retraction of the restraint as the stent flips into an inverted state, and FIG. 2J shows the inverting stent in its inverted position and the restraint being removed.

FIGS. 2K-N is a partial sectional view illustrating another deployment method of the stent-graft of FIG. 1A, where FIG. 2K shows a portion of a delivery device without the stent-graft and delivery catheter; FIG. 2L is a partial sectional view showing the stent-graft of FIG. 1A loaded within a retractable sheath and coupled to the device of FIG. 2K in a pre-deployment unretracted position, FIG. 2M is a partial sectional view of the stent-graft delivery system of FIG. 2L with the retractable sheath partially retracted and the inverting or invertible stent partially released, and FIG. 2N is a partial sectional view of the stent-graft delivery system of FIG. 2M after deployment of the inverting or invertible stent where the leading end of the inverting or invertible stent has been released and the inverting stent (hidden from view) has returned to a position as shown in FIG. 1A.

FIGS. 3A and 3B diagrammatically illustrate another deployment method of the stent-graft of FIG. 1A without an end restraint and with optional hooks, where FIG. 3A shows the inverting stent deployed and expanded with the optional hooks penetrating the vessel wall where the stent-graft is being deployed, and FIG. 3B illustrates the inverting stent self-inverting to return toward its free state where it has pulled the end of the stent-graft tubular graft attached thereto therein to a position similar to that shown in FIG. 1F.

FIGS. 4A, 4B1, 4B2, and 4C diagrammatically illustrate another embodiment according to the invention, where FIG. 4A shows a stent-graft with its invertible stent in a free or unrestrained state, FIG. 4B1 shows the stent graft of FIG. 4A with the invertible stent inverted, FIG. 4B2 shows the stent-graft of FIG. 4B2 radially compressed and loaded and restrained in a delivery sheath, and FIG. 4C shows the inverted stent returning to its free state after sheath removal.

FIG. 4D is an end view of a variation of the stent-graft of FIG. 4A where the stent-graft is the same except for having a different number of apexes.

FIG. 4E is an end view of another variation of the stent-graft of FIG. 4A where the stent-graft is the same except for having a different number of apexes.

FIG. 4F is a variation of the embodiment of FIG. 4E where material is provided between the invertible stent undulations to enhance sealing aspects of the device.

FIG. 5A diagrammatically shows the stent-graft of FIG. 4A extending from the aorta through a fenestration in another stent-graft and into the left subclavian artery and providing a sealing engagement with the fenestrated stent-graft.

FIG. 5B diagrammatically shows the stent-graft of FIG. 4A extending through a fenestration in another stent-graft and into a renal artery from the abdominal aorta and providing a sealing engagement with the fenestrated stent-graft.

### DETAILED DESCRIPTION

The following description will be made with reference to the drawings where when referring to the various figures, it should be understood that like numerals or characters indicate like elements. Further, when referring to catheters, delivery devices, and loaded fasteners described below, the proximal end is the end nearest the operator and the distal end is farthest from the operator when referring to the implanted device.

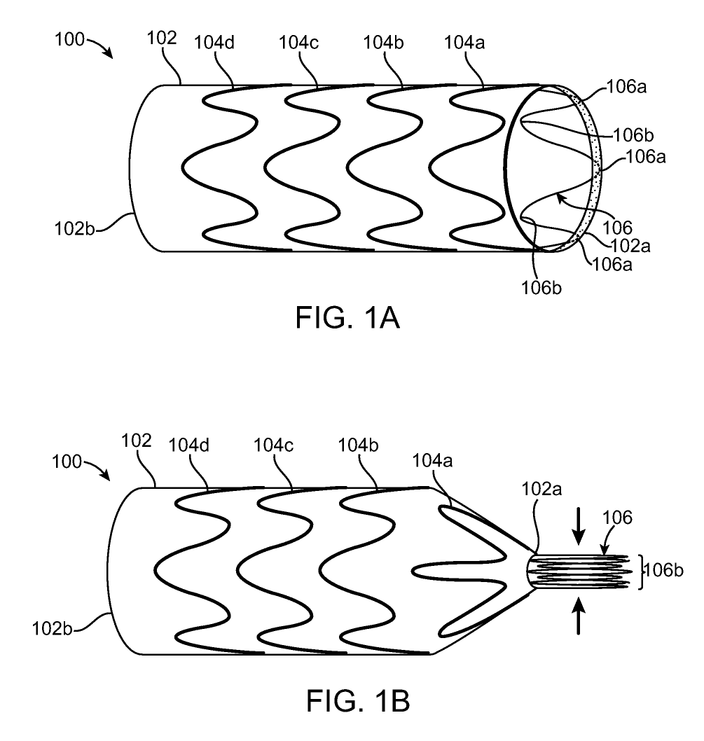

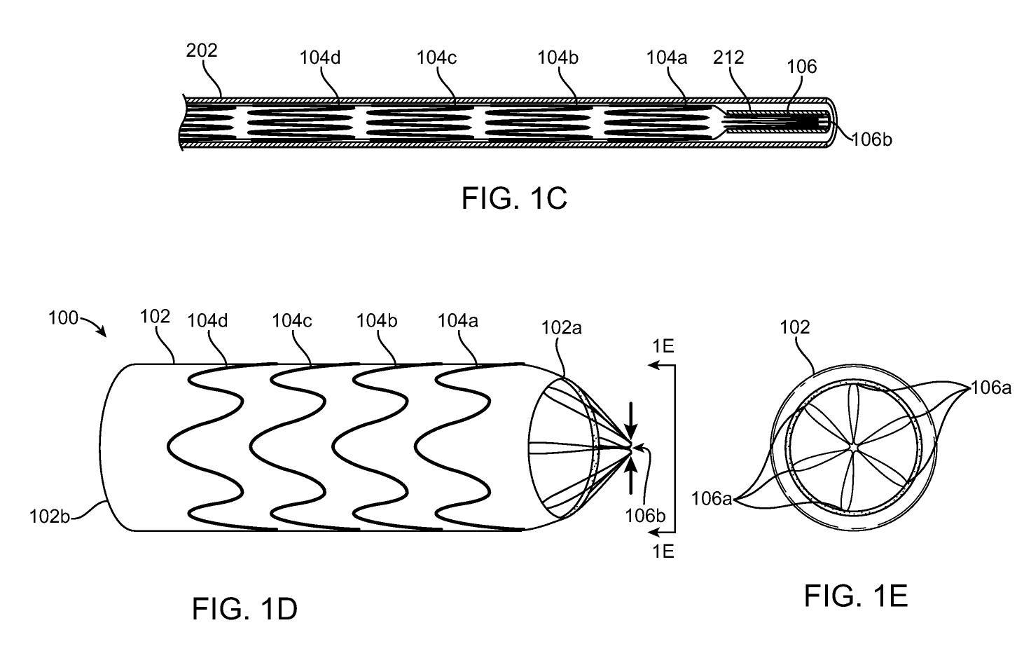

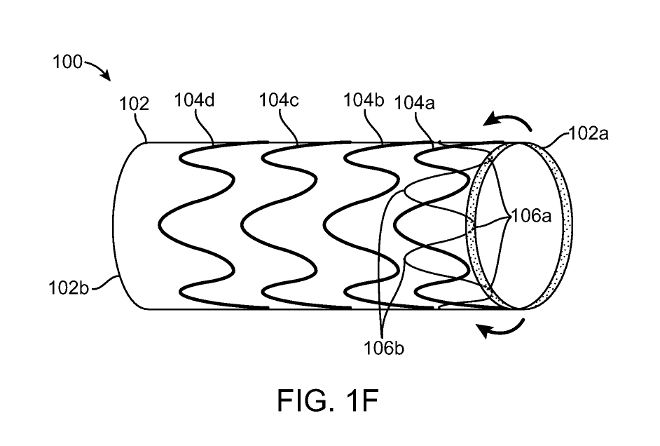

In one embodiment according to the invention, a tubular prosthesis includes a tubular graft and an undulating stent ring secured to the graft in a manner such that it can be inverted to extend forward of the tubular prosthesis for delivery to a desired site in a lumen of a patient and then allowed to move back to an uninverted state where is rests either against the inner surface or outer surface the tubular graft. In the example illustrated in FIGS. 1A-F, the diametrical configuration of the undulating stent ring is controlled to control movement of the undulating stent back to one of an uninverted state where it rests against the inner surface of the tubular graft and an uninverted state where it rests against the outer surface of the tubular graft.

Referring to FIG. 1A, stent-graft 100 is a self-expanding stent-graft and comprises a tubular graft 102, which can be made from any conventional graft material such Dacron® or expanded polytetrafluoroethylene (ePTFE), and one or more conventional stents and an inverting or invertible stent. In the illustrative example, undulating annular stents 104a-d are shown secured to the outer surface of tubular graft 102 and an inverting or invertible stent 106 is shown inside tubular graft 102 along an inner surface thereof. Stents 104a-d can be secured to the graft using sutures or other conventional means and in this example are secured such that they do not move away from the graft surface unlike inverting or invertible stent 106 as will be described in more detail below. In one alternative embodiment, stents 104a-d can be positioned on the interior of the graft member and secured thereto in the same manner.

Tubular graft 102 has a first (or leading) end 102a and a second (or trailing) end 102b and a central portion therebetween. Inverting or invertible stent 106 is secured to end 102a. More specifically, stent 106 comprises an undulating wire having a plurality of apexes and being formed in a closed ring configuration. A first end of stent 106 is defined by apexes 106a and the other end of stent 106 is defined by apexes 106b. In the illustrative embodiment, only apexes 106a are secured to tubular graft 102 so as to form a circumferentially oriented hinge about which stent 106 can be pivoted and/or inverted to the configuration shown in FIG. 1B. In contrast, stents 104a-d are sutured or otherwise secured along their entire length to the tubular graft 102 or substantially along their entire length to tubular graft 102 without such a hinge. In the embodiment shown in FIG. 1A, a portion of first end (or end margin) 102a is folded over a portion of apexes 106a and secured to the tubular graft material with any suitable means such as sutures or glue. In an alternate embodiment, apexes 106a can be directly sutured to the inner surface of tubular graft 102 and the graft not folded back thereover. A plurality of sutures or suture loops can be used at each apex 106a or a single suture loop used at each apex. The multiple sutures or suture loop arrangement or single suture loop arrangement can be made to provide some slack so that the suture can slide along stent 106 and pull the end of tubular graft therewith. In further variations, apexes 106a can be secured to the outer surface of tubular graft 102 using any of the securing approaches described above.

Referring to FIGS. 1B and 1C, loading of stent-graft 100 will be described. First, the free end of stent 106 or apexes 106b are pulled outwardly and the stent pivots about the hinge formed between apexes 106a and tubular graft 102 to the position shown in FIG. 1B, thereby inverting stent 106. The inverted stent extends forwardly of tubular graft end 102a and is radially compressed to lie in the position shown in FIG. 1B with apexes 106b pointing away from the central portion of the stent-graft and then the inverting or invertible stent is restrained in restraint tube 212. The remainder of the stent-graft is radially compressed and the stent-graft is loaded in tubular sheath 202 which restrains stent-graft 100 in a radially compressed state with stent 106 in its inverted state where it is like a loaded spring tending to return to its uninverted state alternate configurations of which are shown in FIGS. 1A and 1F.

Referring to FIGS. 1D and 1E, when stent-graft 100 is deployed such that stent apexes 106b are released after sufficient radial expansion of tubular graft 102, stent 106 returns to the inner surface of stent-graft 100 as shown in FIG. 1A where it applies a radial force against the inner surface of the stent due to its preshaped configuration. For example, the free-state diameter of stent 106 can be slightly greater than tubular graft 102. Alternatively, stent 106 can be deployed in a manner (e.g., where the tubular graft is not allowed to radially expand before release of stent 106) such that it returns to the outer surface of tubular graft 102. For example, apexes 106b can be deployed from sheath 202 such they will fold back around the outer surface of tubular graft 102 as shown in FIG. 1 F. Stent 106 also can include optional hooks to assist in anchoring the device as will described in more detail below.

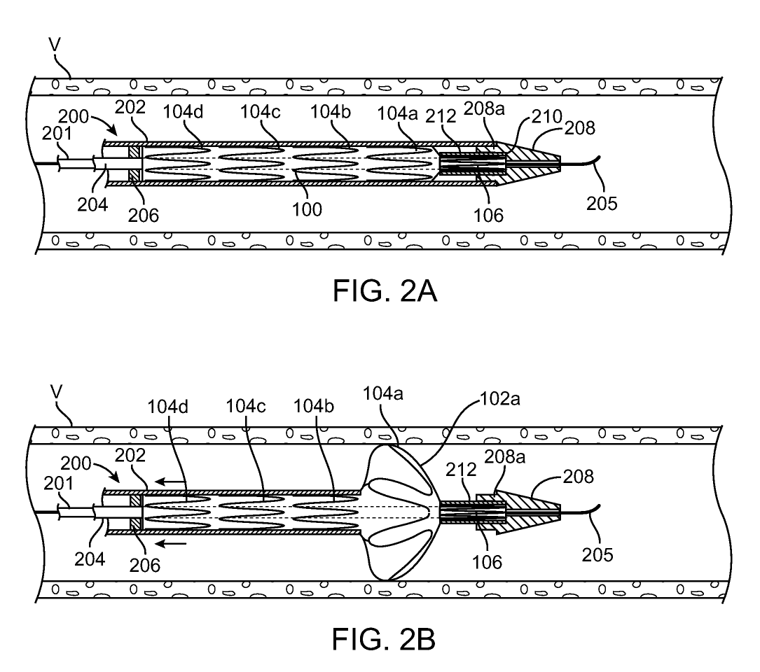

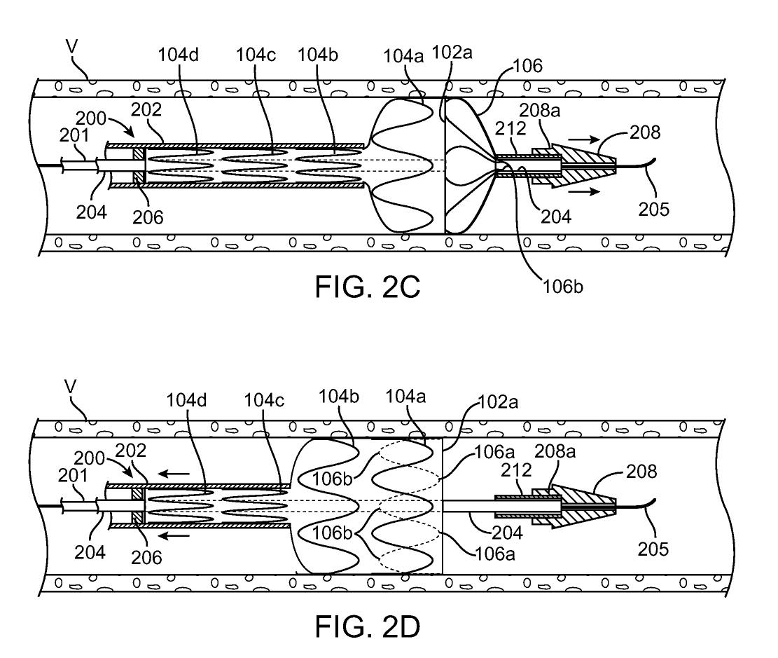

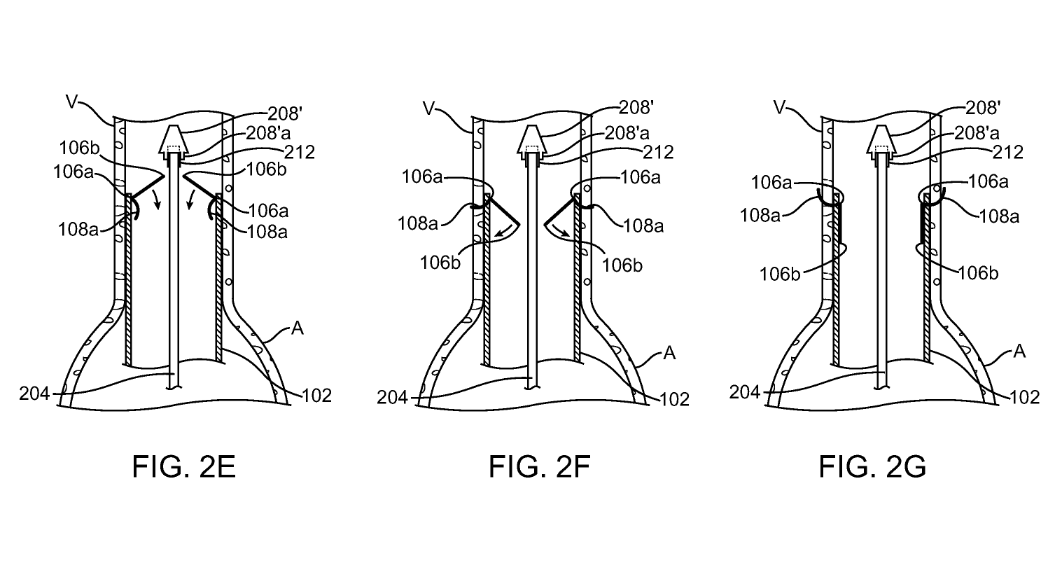

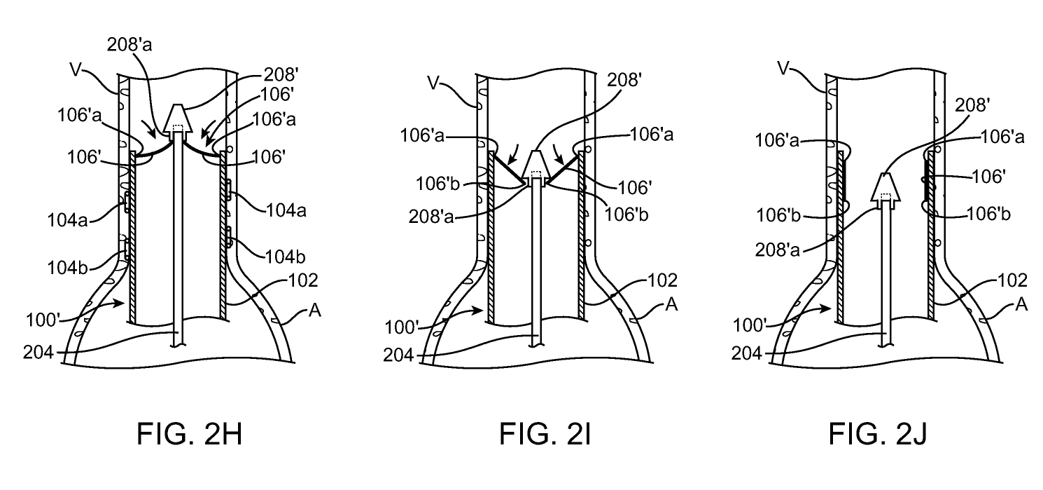

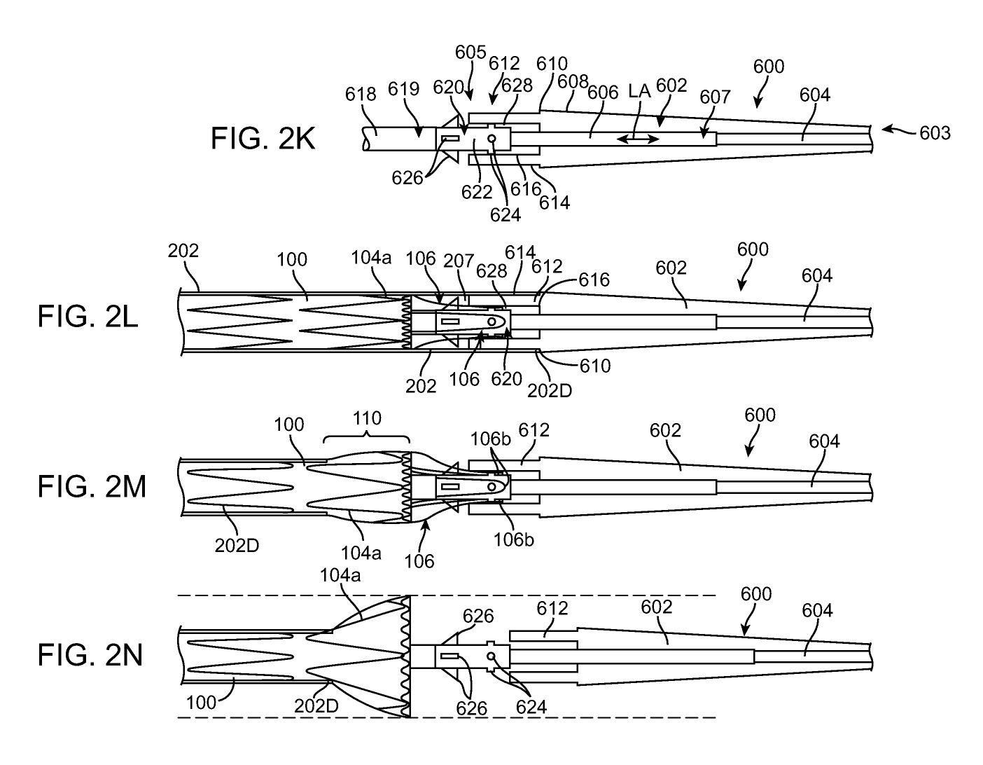

Referring to FIGS. 2A-D, one embodiment of a stent-graft delivery system is shown in a pre-deployment loaded state FIG. 2A and three partial deployment states (FIGS. 2B, 2C, and 2D) and generally designated with reference numeral 200. Delivery catheter system 200 includes catheter or sheath 202, which can be referred to as an outer tube, middle member 204, and inner guidewire tube 201, which tracks over guidewire 205. Sheath 202, middle member 204, and guidewire tube 201 are coaxial and arranged for relative axial movement therebetween. Stent-graft 100 is radially compressed, with stent 106 inverted along its entire length (or along substantially its entire length in the case where apexes 106a are not flipped inside out) and positioned within the distal end of outer tube 202 in front of pusher member or stop 206, which is concentric with and secured to inner middle member 204 and can have a disk or ring shaped configuration with a central access bore to provide access for guidewire tube 201 therethrough. Inverted stent 106 is held or retrained in tube 212, which is positioned within outer tube 202 and extends into tapered tip 208. For purposes of simplification, stent-graft 100 is shown with four undulating stent members 104a-d. A radiopaque ring can be provided on the inside of the distal end portion of catheter or sheath 202 adjacent to the tapered tip 208 to assist with imaging the distal end of sheath 202 using fluoroscopic techniques. Tapered tip 208 has a tubular reduced diameter section 208a, which forms a sleeve over which the distal end of catheter or sheath 202 is positioned. Catheter sheath 202 and reduced diameter section 208a are sized so as to provide a friction fit therebetween that can be readily decoupled when, for example, tapered tip 208 is held in a fixed position and catheter or sheath 202 retracted. However, the inner diameter of reduced diameter section 208a can be sized slightly smaller than the outer diameter of restraint tube 212 such that after restraint tube 212 is positioned therein during assembly, restraint tube 212 remains in tapered tip 208 when catheter 202 is retracted due to a relatively tighter fit between restraint tube 212 and the inner wall of reduced diameter section 208a.

Once the catheter of the delivery system 200 is positioned at the desired site for deployment of the prosthesis, the middle member 204 with stop 206 and the guidewire tube 201 are held stationary and the outer tube, catheter or sheath 202 withdrawn so that the proximal end of the stent-graft is gradually exposed and allowed to expand. Tapered tip 208 has an annular recess or cavity 210 in which a portion of tubular restraint 212 is positioned, acting as a restraint restrains apexes 106a as described above. Stop 206 therefore is sized to engage the distal end of the stent-graft as the stent-graft is deployed. The proximal ends of the sheath 202, middle member 204 and guidewire tube 201 are coupled to and manipulated by a handle suitable for a physician or interventionalist's manipulation as is known in the art. Restraint tube 212 is configured to retain the apexes 106a in a radially compressed configuration before allowing expansion thereof during a later phase of their deployment. Alternatively, any of the stent-graft deployment systems described in co-owned U.S. Pat. No. 7,264,632 to Wright et al. and is entitled Controlled Deployment Delivery System, the disclosure of which is hereby incorporated herein by reference in its entirety, can be incorporated into stent-graft delivery system 200. Other stent-graft delivery systems that can be used include the Endurant® stent-graft delivery system manufactured by Medtronic, Inc. (Minneapolis, Minn.), which is described in co-owned U.S. patent application Ser. No. 11/559,754 to Mitchell et al, filed Nov. 14, 2006 and entitled Delivery System for Stent-Graft With Anchoring Pins, the disclosure of which is hereby incorporated herein by reference in its entirety.

Referring to FIG. 2B, catheter sheath 202 is shown partially pulled back and a portion of the prosthesis partially expanded. In this partially retracted position, the proximal end of the prosthesis is constrained allowing the prosthesis to be repositioned (e.g., longitudinally or rotationally moved) if desired before release of the proximal end of the prosthesis. The surgeon or interventionalist can determine if prosthesis repositioning is desired based on monitored movement of the prosthesis using fluoroscopy during deployment, which will be described in more detail below.

Referring to FIG. 2C, after a sufficient length of the stent-graft 100 has expanded, sheath 202 and middle member 204 are held stationary and guide tube 201, which is fixedly secured to tapered tip 208, which also tracks over guidewire 205, is advanced to further separate tapered tip 208 from catheter sheath 202 and to release a portion of stent 106 and allow stent 106 to start to expand. As the tapered tip is further advanced, tubular restraint 212 releases apexes 106b (FIG. 2D). Inverted stent 106 then flips back to its uninverted state as shown in dashed line where it is inside tubular graft 102 and applies a radial outward force through the graft material against vessel “V” to provide an increased force to anchor the stent-graft. In this regard, stent 106 can be provided with a predetermined configuration to enhance its ability to apply such outwardly directed radial pressure against the tubular graft and vessel wall. In one embodiment, stent 106 can be preformed with a conical or tapering shape to provide or accentuate such radial pressure

FIGS. 2E-G diagrammatically illustrate deployment of the inverted stent of FIG. 1A with optional hooks 108a extending from apexes 106a, which are held in tapered tip 208′. Tapered tip 208′ is diagramatically shown in FIGS. 2E-G and can have the same construction as tapered tip 208 and, thus, can include a reduced diameter tubular section 208′a similar to catheter or sheath receiving reduced diameter section 208a shown in FIGS. 2A-D. FIG. 2E shows the stent 106 released from restraint tube 212 where it was restrained in its inverted and radially compressed state such as the state shown in FIG. 1B. FIG. 2F shows stent 106 moving inside the stent-graft 100 and hooks 108a penetrating the stent-graft. FIG. 2G shows stent 106 after it has returned to a position similar to that shown in FIG. 1A along the inner surface of the stent-graft with hooks 108a fully engaged through the portion of the wall of vessel “V” above aneurysm “A.”

FIGS. 2H-J diagrammatically illustrate deployment of another embodiment according to the invention which comprises a stent-graft 100′ including a tubular graft such as tubular graft 102 and can include stents incorporated therein in the same manner as stents 104a,b,c,d are incorporated in stent-graft 100 described above. For purposes of example, stents 104a and 104b are shown in FIG. 2H. In this embodiment, however, the invertible stent (invertible stent 106′) is (1) inverted when inside tubular graft 102 and (2) in a free state when outside tubular graft 102 and otherwise not radially constrained with a delivery sheath or tapered tip and its tubular restraint. Otherwise, stents 106 and 106′ are the same. Stent 106′ includes apexes 106′a and apexes 106′b which correspond to apexes 106a and 106b. Apexes 106′a are pivotally connected to the distal end 102a of tubular graft 102a along the perimeter of tubular graft end 102a′, or the inner or outer surface of tubular graft 102 to provide a single attachment point for each apex 106′a that acts as a hinge point. The attachment of all of apex's 106′a to tubular graft 102 collaboratively creates a set of circumferentially arranged hinge points about which one end of stent 106′ can pivot. The hinges can be formed using a single suture loop extending about each apex 106′a and through the tubular graft. In further alternatives, apexes 106′a can be sandwiched between tubular graft 102 and another annular ring of graft material placed on the inner surface tubular graft 102 if apexes are placed on the inner surface of tubular graft 102 or the outer surface of tubular graft 102 if the apexes are placed on the outer surface of tubular graft 102. With this construction, apexes 106′a can be urged inside the stent-graft 100′ from a position outside the stent-graft to have stent 106′ reside within the stent-graft in an inverted configuration where it provides an outward radial force. Any suitable mechanism can be used to urge apexes 106′b to an inverted position inside the stent-graft. Stent-graft 100′ is placed at the target site in a vessel and either fully or partially deployed. Stent 106′ is then pushed or pulled into the interior of tubular graft 102. This can be done with any suitable means.

In the example illustrated in FIG. 2H, tapered tip or stent apex restraint 208′ is retracted after partial release of the stent 106′ where stent apexes 106′b remain inside tapered tip sleeve section 208′a. Tapered tip 208′ is retracted to pull and pivot the invertible stent 106′ about its circumferentially oriented hinge points to a position within the tubular graft 102. Tapered tip 208′ can have the same construction as tapered tip 208 and, thus, can include a reduced diameter section 208′a similar to catheter or sheath receiving reduced diameter section 208a shown in FIGS. 2A-D. FIG. 2I shows further retraction of restraint 208′ and FIG. 2J shows stent 106′ in its inverted position where as a result of its spring properties it applies a radial outward force against the wall of vessel “V” above aneurysm “A.” Restraint 208′ is then removed. If desired, a separate expansion member such as a balloon mounted on a separate balloon catheter can be used to radially expand stent 106′.

Other inverting mechanisms also may be used. For example, a pull wire or suture can be secured to each apex 106a and each wire or suture extended back through catheter 202. Stent-graft 100′ is deployed so that stent 106′ extends outside and beyond tubular graft 102 and generally parallel to the end portion from which it extends. Accordingly, stent 106′ is outside tapered tip 208′ and extending along the inner wall of vessel “V”. The wires or sutures are pulled to pull stent apexes 106′a into tubular graft 102 such that the stent is in an inverted state in the tubular graft. In this case, the tapered tip 208′ is not used to pull stent 106′ inward, but can be, for example, advanced to release stent 106′ as shown, for example, in FIGS. 2B or 2E, after which the wires or sutures are pulled to invert stent 106′. The wires or sutures then can be cut using a conventional endolumenal wire/suture cutting mechanism. In a further variation, wires or sutures can be used where one end of each wire or suture is secured to an apex 106′b and the other end of the wire or suture secured to tapered tip 208′, for example, at the trailing end of sleeve 208′a, which is opposite the leading end of tapered tip 208′. After stent 106′ is fully released and positioned along the inner wall of vessel “V,” tapered tip is retracted to pull stent inside stent-graft 100 to a position as shown in FIG. 2J. The wires or sutures then can be cut as described above.

Regarding stent 106′, any other suitable delivery apparatus also can be used such as apparatus described in U.S. patent application Ser. No. 12/052989 to Brian Glynn filed on 21 Mar. 2008, the disclosure of which is hereby incorporated by reference herein in its entirety.

FIG. 2K is a partial cross-sectional view of a portion of another stent-graft delivery system that can be used and which is shown without a stent-graft and outer sheath. The mechanism shown in FIG. 2K is described in co-owned U.S. patent application Ser. No. 11/559,754 to Mitchell et al, filed Nov. 14, 2006 and entitled Delivery System for Stent-Graft With Anchoring Pins, the disclosure of which is hereby incorporated herein by reference in its entirety. Stent-graft delivery system 600 includes a tapered tip 602 that is flexible and able to provide trackability in tight and tortuous vessels. Tapered tip 602 includes a guidewire lumen 604 therein for connecting to adjacent members and allowing passage of a guidewire through tapered tip 602. Other tip shapes such as bullet-shaped tips could also be used.

An inner tube 606 defines a lumen, e.g., a guide wire lumen, therein. A distal end 607 of inner tube 606 is located within and secured to tapered tip 602, i.e., tapered tip 602 is mounted on inner tube 606. As shown in FIG. 2K, the lumen of inner tube 606 is in fluid communication with guidewire lumen 604 of tapered tip 602 such that a guide wire can be passed through inner tube 606 and out distal end 607, through guidewire lumen 604 of tapered tip 602, and out a distal end 603 of tapered tip 602.

Tapered tip 602 includes a tapered outer surface 608 that gradually increases in diameter. More particularly, tapered outer surface 608 has a minimum diameter at distal end 603 and gradually increases in diameter proximally, i.e., in the direction of the operator (or handle of stent-graft delivery system 600), from distal end 603.

Tapered outer surface 608 extends proximally to a primary sheath abutment surface (shoulder) 610 of tapered tip 602. Primary sheath abutment surface 610 is an annular ring perpendicular to a longitudinal axis “LA” of stent-graft delivery system 600.

Tapered tip 602 further includes a (tip) sleeve 612 extending proximally from primary sheath abutment surface 610. Generally, sleeve 612 is at a proximal end 605 of tapered tip 602. Sleeve 612 is a hollow cylindrical tube extending proximally and longitudinally from primary sheath abutment surface 610. Sleeve 612 includes an outer cylindrical surface 614 and an inner cylindrical surface 616.

Stent-graft delivery system 600 further includes an outer tube 618 having a spindle 620 located at and fixed to a distal end 619 of outer tube 618. Spindle 620 includes a spindle body 622 having a cylindrical outer surface, a plurality of spindle pins 624 protruding radially outward from spindle body 622, and a plurality of primary sheath guides 626 protruding radially outward from spindle body 622. Primary sheath guides 626 guide the primary sheath into position over (tip) sleeve 612 (see FIG. 2L for example).

As illustrated in FIG. 2K, spindle 620 is configured to slip inside of sleeve 612 such that spindle pins 624 are directly adjacent to, or contact, inner cylindrical surface 616 of sleeve 612. Spindle pins 624 extend from spindle body 622 towards and to sleeve 612. Generally, the diameter to which spindle pins 624 extend from spindle body 622 is approximately equal to, or slightly less than, the diameter of inner cylindrical surface 616 of sleeve 612 allowing spindle pins 624 to snugly fit inside of sleeve 612. An annular space 628 exists between inner cylindrical surface 616 and spindle body 622.

Inner tube 606 is within and extends through outer tube 618 and spindle 620. Inner tube 606 and thus tapered tip 602 is moved along longitudinal axis L (longitudinally moved) relative to outer tube 618 and thus spindle 620 to release the proximal end of a stent-graft as discussed further below.

FIG. 2L is a partial cross-sectional view of the stent-graft delivery system 600 of FIG. 2K including a stent-graft 100 located within a retractable primary sheath 202 in a pre-deployment un-retracted position.

Primary sheath 202 is a hollow tube and defines a lumen 207 therein through which outer tube 618 and inner tube 606 extend. Primary sheath 202 is in a pre-deployment un-retracted position in FIG. 2L. Primary sheath 202 is moved proximally along longitudinal axis “LA,” sometimes called retracted, relative to outer tube 618/spindle 620 and, thus, stent-graft 100 to deploy a portion of stent-graft 100 as discussed further below. As described above, stent-graft 202 can be a self-expanding stent-graft such that it self-expands upon being released from its radially constrained position. In accordance with this example, stent-graft 100 includes tubular graft 102 and support structures (stents 104a-d) and inverting or invertible stent 106 attached to the tubular graft as discussed above. Tubular graft 102 includes a proximal or leading end 102a and a distal or trailing end 102b.

As shown in FIG. 2L, stent-graft 100 is in a radially constrained configuration over outer tube 618 and spindle 620. Stent-graft 100 is located within and radially compressed by primary sheath 202. Inverting or invertible stent 106 is radially constrained and held in position in annular space 628 between spindle body 622 and inner cylindrical surface 616 of sleeve 612.

Generally, the graft material of stent-graft 100 is radially constrained by primary sheath 202 and the leading portion of inverting or invertible stent 106 is radially constrained by sleeve 612 allowing sequential and independent deployment of the graft material and inverting or invertible stent 106 of stent-graft 100.

Primary sheath 202 includes a distal end 202D adjacent to or in abutting contact with primary sheath abutment surface 610 of tapered tip 602. Distal end 202D fits snugly around sleeve 612 and in one example lightly presses radially inward on outer cylindrical surface 614 of sleeve 612.

FIG. 2M is a partial cross-sectional view of the stent-graft delivery system 600 of FIG. 2L with retractable primary sheath 202 partially retracted. Referring now to FIG. 2M, primary sheath 202 is partially retracted such that distal end 202D is spaced apart from tapered tip 602. Further, due to the retraction of primary sheath 202, a proximal portion 110 of stent-graft 100 is exposed and partially deployed.

As proximal portion 110 is only partially deployed and a portion of inverting or invertible stent 106 is radially constrained and un-deployed, stent-graft 100 can be repositioned in the event that the initial positioning of stent-graft 100 is less than desirable. More particularly, to reposition stent-graft 100, the retraction of primary sheath 202 is halted. Stent-graft delivery system 600 is then moved to reposition stent-graft 100, for example, stent-graft 100 is rotated or moved proximally or distally without a substantial risk of damaging the wall of the vessel in which stent-graft 100 is being deployed.

Further, as inverting or invertible stent 106 is secured and in kept in tension as primary sheath 202 is retracted and, in one example, the distal end of the stent-graft (not shown) is free to move within primary sheath 202, bunching of stent-graft 100 during retraction of primary sheath 202 is avoided. By avoiding bunching, frictional drag of stent-graft 100 on primary sheath 202 during retraction is minimized thus facilitating smooth and easy retraction of primary sheath 202.

Once stent-graft 100 is properly positioned, apexes 106b are released to allow inverting or invertible stent to return to the inside of stent-graft 100 as discussed above (see e.g., FIG. 1A).

FIG. 2N is a partial cross-sectional view of the stent-graft delivery system 600 of FIG. 2M after deployment of inverting or invertible stent 106. Referring now to FIG. 2M, tapered tip 602 is advanced relative to spindle 620 to expose the proximal end of apexes 106b of stent 106 so that stent 106 (hidden from view) returns to its position inside stent-graft 100 as described above (see e.g., FIG. 1A). If necessary, spindle 620 can be retracted within stent-graft 100 to provided clearance for stent 106 to return to its position inside the stent-graft such as shown in FIG. 1A.

In another example, primary sheath 202 is fully retracted prior to release of inverting or invertible stent 106. To illustrate, instead of being partially retracted at the stage of deployment illustrated in FIG. 2M, primary sheath 202 is fully retracted while the stent 106 is still radially constrained.

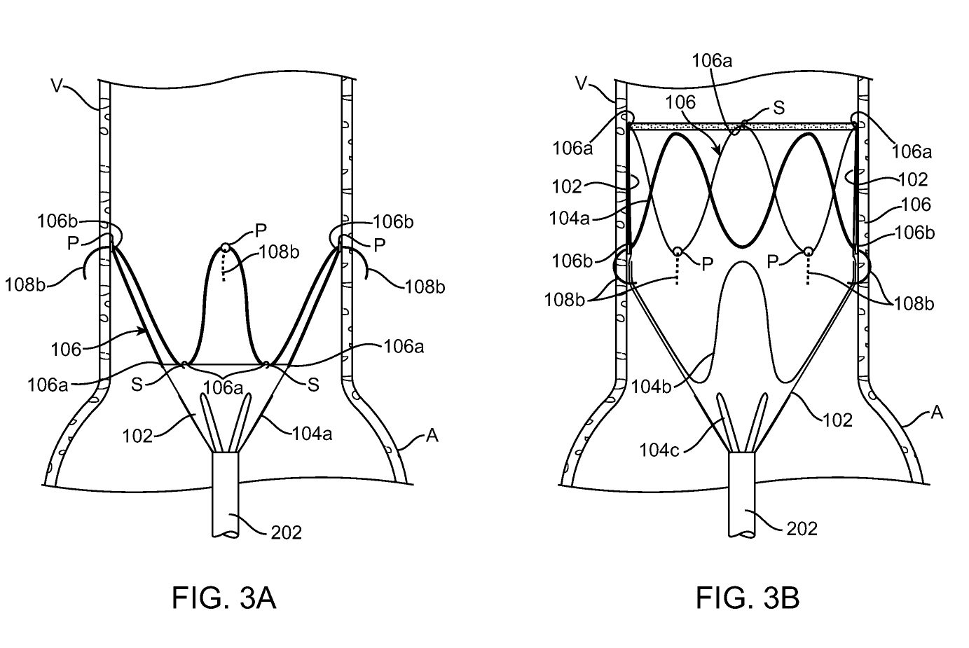

FIGS. 3A and 3B diagrammatically illustrate another deployment method of the stent-graft of FIG. 1A without an end restraint 212 (or with restraint 208′ advanced prior to stent-graft deployment) and with optional hooks 108b extending from apexes 106b. FIG. 3A shows the inverting stent after it has been deployed from sheath 202. It is in an expanded state with hooks 108b penetrating vessel “V” above aneurysm “A.” FIG. 3B illustrates the inverting stent after it has self-inverted and returned to a position along the outer surface of tubular graft 102 as shown in FIG. 1F. As inverting stent 106 self-inverts, it pulls tubular graft end 102a into the inverting stent to the position shown in FIG. 3B. Pledgets “P” can be provided at the base of hooks 108b minimize or eliminate the risk of blood flow driving apexes 106b into the vessel wall. The number of apexes in the inverting or invertible stent can vary depending of the application or as desired. For example, four to eight apexes 106a can be used with a corresponding number of apexes 106b. However, an inverting or invertible stent having more or fewer apexes also can be used.

Although a non-bifurcated stent-graft configuration has been shown, the inverting or invertible stent described herein can be used in bifurcated stent-grafts where they typically will be positioned along end opposite the bifurcation (e.g., along the distal end of an AAA bifurcated stent-graft. Other configurations including more or fewer stents 104 or bifurcated constructions can be used. For example, a bifurcated stent can be provided with an inverting or invertible stent at its distal end and otherwise only one stent at its other ends, thereby enabling a reduced profile when radially compressed for delivery.

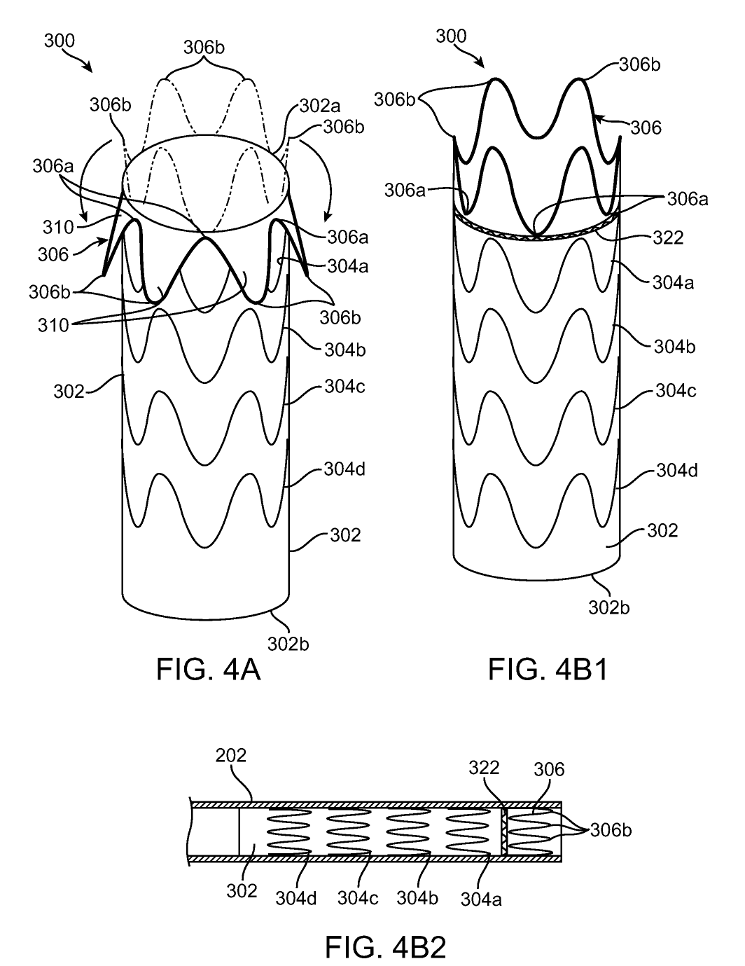

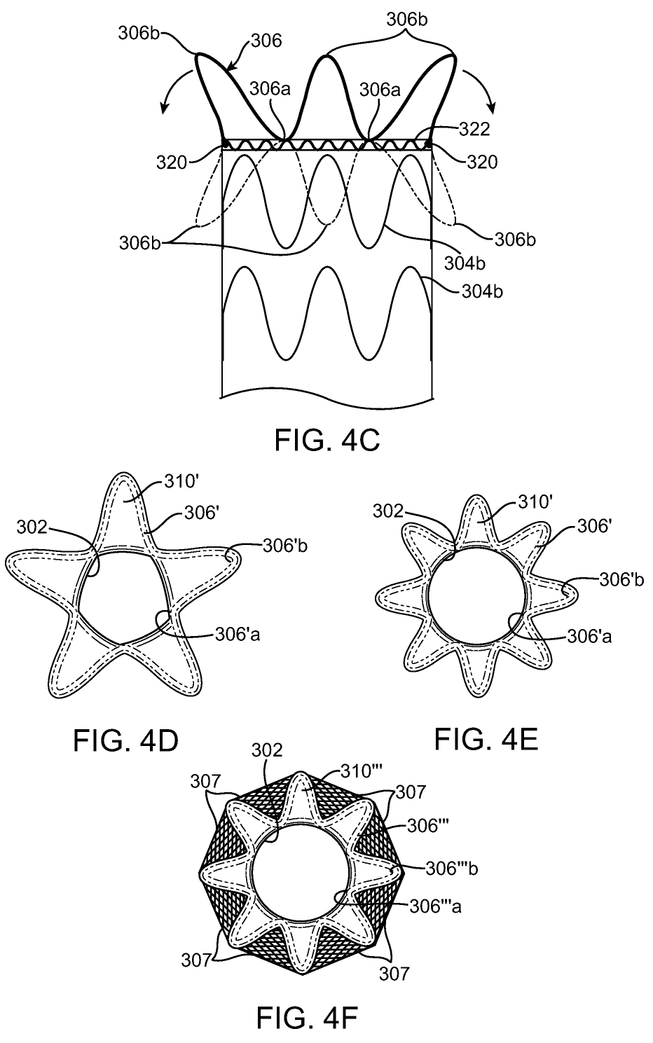

Referring to FIGS. 4A, 4B1, 4B2 and 4C, another self-expanding stent-graft with an inverting or invertible stent is shown in accordance with the principles of the embodiments presented. This embodiment addresses challenges with securing a branch vessel covered stent in a fenestration of another stent-graft in situ. According to this embodiment, an inverting or invertible stent is provided in a branch vessel stent-graft at proximal end of the stent-graft for thoracic aortic aneurysm applications or the distal end in abdominal aortic aneurysm applications. The inverting or invertible stent provides the stent-graft with the ability to fold back onto itself when deployed and to engage the area of the fenestrated stent-graft around the fenestration. The stent-graft also can have visual markers (e.g., radiopaque markers) to aid in the optimal placement of the inverting or invertible stent adjacent to the fenestration. With this construction the risk of one or more of the following can be reduced: tear propagation in the fenestrated stent-graft, stent-graft migration, and leakage between joined stent-grafts at branch vessels.

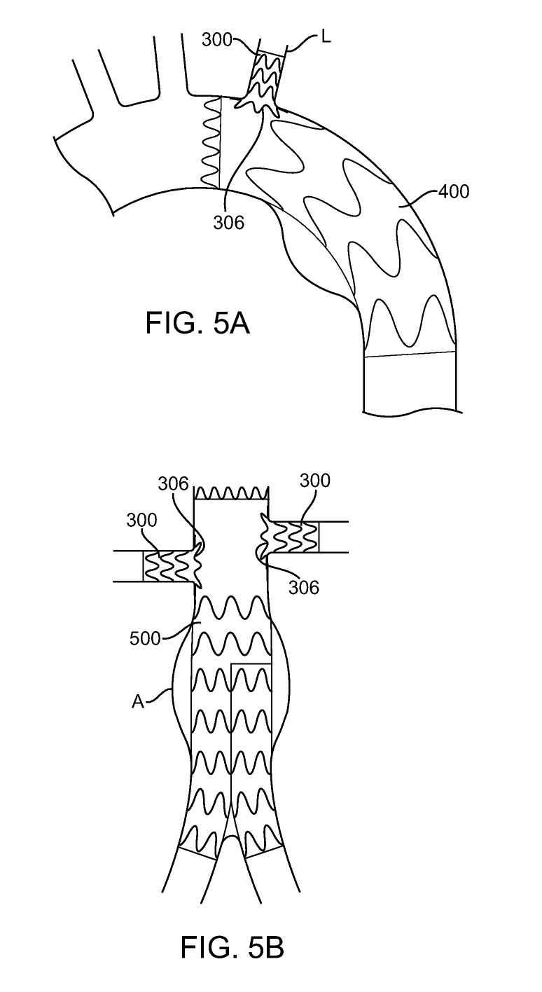

Referring to FIG. 4A, stent-graft or covered stent 300, which can be a self-expanding stent-graft, includes tubular graft 302, which has a first end 302a and a second end 302b and a central portion therebetween. Covered stent 300 further includes a plurality of stents (e.g., 302a, 302b, 302c, and 302d), which can be secured to tubular graft 302 in the same manner as stents 104a-d are secured to tubular graft 102. Undulating inverting or Invertible stent 306 has apexes 306a at one end and apexes 306b at the other end. Apexes 306a are pivotally secured to tubular graft 302 and can be so secured in the same manner that stent apexes 106a are secured to tubular graft 102. Inverting or invertible stent 306 can be covered with graft material 310 as indicated in FIG. 4A to form a sealing element when it springs back toward the inner surface of the fenestrated stent-graft in which it is positioned. In this regard, inverting or invertible stent 306 can be referred to as a sealing element. Blood flow against the outer surface of the sealing element can enhance the seal. Alternatively, the graft covering for inverting or invertible stent 306 can be integrally formed with tubular graft 302 during manufacture. FIG. 4A shows inverting or invertible stent 306 after it has returned from an inverted state to a free state and having a flower-like configuration. Inverting or invertible stent 306 can be constructed such that its undulations or petals flower and fold back toward graft end 302b when unrestrained up to 180 degrees from the position shown in FIG. 4B1 to allow for significant contact between inverting or invertible stent 306 and/or its graft material 310 and the surface that stent 306 and/or its graft material 310 engages (depending on whether the stent is secured to the inner surface or outer surface of the graft material) as diagrammatically shown for purposes of example in FIGS. 5A and 5B.

Referring to FIG. 4B1 stent graft (covered stent) 300 is shown with optional spring coil 322 to provide radial support as is known in the art and radiopaque markers 320.

Referring to FIG. 4B2, stent-graft 300 is radially compressed and restrained in catheter 202 with apexes 306b directed away from the central portion of tubular graft 302.

FIG. 4C illustrates inverting or invertible stent 306 springing back toward its free state after sheath 202 has been removed.

Inverting or invertible stent 306 is shown with a six petaled configuration with six apexes 306b in FIGS. 4A-C and as best seen in FIG. 4B1. However, more or fewer apexes can be used. For purposes of example, a five petaled configuration is shown in FIG. 4D and an eight petaled configuration is shown in FIG. 4E where the inverting or invertible stents are indicated with numerals 306′ and 306″ with apexes 306′a, 306′b and 306″a, 306″b, respectfully. The graft covering 310′ or 310″ for inverting or invertible stents 306′ and 306″ can be integrally formed with tubular graft 302 during manufacture as noted above.

FIG. 4F shows and alternate embodiment of that shown in FIG. 4E, where the spaces between the petals are covered by graft material 307 and sized to remain substantially loose when the invertible stent 306′″ is oriented at approximately 90 degrees from the centerline axis of the covered stent 300. Otherwise the embodiment of FIGS. 4E and 4F are the same where invertible stent 306″ has the same construction as stent 306′″ with corresponding apexes 306″a and 306″a′ and 306″b and 306″b, and graft cover 310″ has the same construction as graft cover 310′″. The configuration in FIG. 4F provides the opportunity for the blood pressure to urge the substantially loose material or sections 307 against the stent-graft surface or vessel wall against which the stent apexes 306′″b (or their graft cover) are in contact with the adjacent structure. Material or webbing 307 can be any suitable material such as a fabric that provides a barrier to blood flow and can be foldable or stretchable material and can be sewn to graft material 310′″ between adjacent undulations of undulating invertible stent 306′″.

Referring to FIG. 5A, a thoracic delivery application is shown. Main stent-graft 400, which can include a plurality of stents similar to stent 104a-d, is shown positioned within the aorta to bypass an aneurysm and fenestrated to provide access to the left subclavian artery “L.” Branch covered stent 300 is delivered to the site while being restrained in sheath 202 and passed through the fenestration and into the left subclavian artery. The sheath is retracted and covered stent 300 deployed. Inverting stent 306 self-inverts or springs back toward the central portion of tubular graft 302 such that stent 306 or its cover sealingly engages the inner surface of stent-graft 400 around the fenestration.

Referring to FIG. 5B, covered stent 300 is delivered via sheath 202 through a fenestration in bifurcated stent-graft 500, which can include a plurality of stents similar to stent 104a-d and is positioned within the abdominal aorta to bypass an aneurysm “A.” Stent-graft 500 has a fenestration on opposite sides to provide access to each of branch vessels BV1 and BV2, which can correspond to the renal arteries. Covered stent 300 is introduced into branch vessel BV1 using catheter sheath 202, which is then retracted to deploy covered stent 300 such that the inverting stent self-inverts or springs back toward the central portion of tubular graft 302 and sealingly engages the inner surface of stent-graft 500 around the fenestration. Another stent-graft 300 is then similarly deployed in branch vessel BV2. Covered stent deployment can in the instances where the covered stent is introduced from the main vessel, be deployed using a hub to tip deployment system and method, where a hub (middle) portion (or proximal end) of the covered stent is first deployed to allow the inverting stent to first emerge and be positioned against the stent graft wall adjacent the side branch opening into which the balance of the covered stent is to be deployed. If a retrograde deployment from outside the main stent graft body into and through the side branch opening is used, normal tip to hub deployment initially deploys the inverting stent to allow it to be correctly positioned before deploying the cylindrical covered stent body.

Further, any of the stents or undulating members described herein can be made from any suitable stent material such as nitinol. The undulating configuration can be provided using conventional techniques where a plurality of pegs are mounted on a flat board in a manner to allow the wire to be wrapped therearound in to form an undulating configuration. The wire is laced about the pegs to form a planar undulating element and the planar undulating element heat treated to heat set it in that configuration to provide a memory set configuration as is known in the art. The ends of the element are secured together with welding or any other suitable means to form a closed ring. In one alternative method for making the inverting or invertible stent 106, the pegs are mounted on a cylindrical mandrel in a manner to allow wrapping the wire in an undulating configuration. The wire is laced about the pegs in an undulating configuration and the ends secured to each other. The undulating ring is then heat treated to provide it with a memory set configuration. This approach provides a greater spring effect for the stent to self-invert to a non-inverted state after having been inverted.

Among the many advantages of the embodiments described herein is low stent-graft delivery profile. More specifically, the inverting or invertible stent can be delivered outside the main body of the stent-graft of which it forms a part.

Any feature described in any one embodiment described herein can be combined with any other feature of any of the other embodiments or features described herein. Furthermore, variations and modifications of the devices and methods disclosed herein will be readily apparent to persons skilled in the art.

What is claimed is:

1. A tubular prosthesis comprising:
a tubular graft having a first end margin, a second end margin and a central portion therebetween; and
an undulating stent having a plurality of apexes, a first end defined at least in part by a first group of said apexes, and a second end defined at least in part by a second group of said apexes, said undulating stent being secured to said tubular graft in a manner such that it can be inverted to extend generally in the same direction as said tubular graft with one end thereof forming an end of said tubular prosthesis and pointing away from said central portion of said tubular graft.

2. The tubular prosthesis of claim 1, wherein said undulating stent forms a closed ring.

3. The tubular prosthesis of claim 1, wherein said undulating stent rests against one of said inner surface and outer surface of said the tubular graft when in an uninverted state.

4. The tubular prosthesis of claim 1, wherein a portion of said undulating stent extends radially when in an uninverted state.

5. The tubular prosthesis of claim 4 wherein said undulating stent folds back toward said central portion when in an uninverted state.

6. The tubular prosthesis of claim 4, wherein said undulating stent is covered with graft material.

7. The tubular prosthesis of claim 6, wherein said graft material forms a portion of said tubular graft.

8. The tubular prosthesis of claim 6, wherein said undulating stent has a star shaped configuration.

9. The tubular prosthesis of claim 1, wherein said undulating stent only is secured to said tubular graft through said first group of said apexes.

10. The tubular prosthesis of claim 1, further including a plurality of sutures securing a plurality of said first group of apexes to said tubular graft material.

11. The tubular prosthesis of claim 10 wherein said sutures are slidable along said undulating stent.

12. The tubular prosthesis of claim 10 wherein only a single suture loop secures each apex of said plurality of said first group of apexes to said tubular graft material.

13. The tubular prosthesis of claim 1, wherein a portion of said first end margin is folded over a portion of said first group of apexes and secured to said tubular graft material.

14. The tubular prosthesis of claim 1, wherein said first group of apexes are secured to the inner surface of said tubular graft.

15. The tubular prosthesis of claim 1, wherein said first group of apexes are secured to said outer surface of said tubular graft.

16. The tubular prosthesis of claim 1, further including a plurality of hooks, each hook extending from one of a plurality of said first group of apexes.

17. The tubular prosthesis of claim 1, further including a plurality of hooks, each hook extending from one of a plurality of said second group of apexes.

18. The tubular prosthesis of claim 1, wherein said tubular prosthesis is a self-expanding stent-graft.

19. A tubular prosthesis delivery system comprising:
a sheath having a distal deployment end and a proximal end;
a radially compressed stent-graft, which has a first end and a second end and is slidably disposed in said sheath and further includes an undulating stent having a plurality of apexes, a first end of the stent being defined at least in part by a first group of said apexes, and a second end of the stent being defined by at least in part by a second group of said apexes, said undulating stent being inverted with said second group of apexes directed toward said distal deployment end of said sheath.

20. The system of claim 19, further including a plurality of hooks extending from said first group of apexes.

21. The system of claim 19, further including a plurality of hooks extending from said second group of apexes.

22. The system of claim 19, wherein said stent-graft is a self-expanding stent-graft.

23. A method of delivering a tubular prosthesis in a vessel in a human patient comprising delivering a tubular prosthesis having an inner surface, an outer surface, and an inverted stent forming the leading end of the prosthesis as it is delivered to a target site in a human vessel and deploying the prosthesis such that the inverted stent folds back over one of the inner and outer surface of the tubular prosthesis.

24. The method of claim 23 wherein a plurality of hooks extend from the inverted stent and pass through the vessel during deployment of the prosthesis.

25. A method of coupling a first tubular prosthesis in a branch vessel to a second tubular prosthesis in a vessel from the branch vessel branches comprising:
delivering a first tubular prosthesis, which is restrained in a sheath and has a leading end and a trailing end, which includes an inverted stent, through a fenestration in a second tubular prosthesis, which is positioned in a first vessel, and into a second vessel that branches from the first vessel;
positioning the inverted stent inside the first tubular prosthesis; and
retracting the sheath to release the first tubular prosthesis and allow the trailing end to move radially outward against an inner surface of the second prosthesis adjacent the branch vessel to form a seal between the first and second prostheses.

26. The method of claim 25 wherein the inverting stent has undulations and is covered with graft material and where webbing extends between the undulations to enhance the seal between the first and second prostheses.

27. A tubular prosthesis comprising:
a tubular graft; and
an undulating stent having a plurality of apexes, a first end defined at least in part by a first group of said apexes, and a second end defined at least in part by a second group of said apexes, said first group of apexes being pivotally attached to said tubular graft so as to form a plurality of circumferentially arranged hinges about which the stent can pivot so that said second group of apexes can move between a position where they are inside the tubular graft and a position where they are outside the tubular graft.
